(19) United States
(12) Patent Application Publication
(10) Pub. No.: US 2018/0334075 A1
(43) Pub. Date: Nov. 22, 2018

(54) Ratchet Type Buckle

(71) Applicant: Buckles International, Inc., Meriden, CT (US)

(72) Inventors: Warren J. Frank, St. Helena Island, SC (US); John L. Morris, Durham, CT (US)

(73) Assignee: Buckles International, Inc., Meriden, CT (US)

(21) Appl. No.: 15/599,832

(22) Filed: May 19, 2017

(51) Int. Cl. B60P 7/08; A61B 17/132

(52) U.S. Cl. CPC B60P 7/083; A61B 17/12; A61B 17/1327; A61B 17/1322

(57) ABSTRACT

A strap buckle features first and second ratchet gears fixed at opposite ends of a slotted axle, for corotation with the axle, a drive gear engaging the first ratchet gear, and a rotationally fixed holding gear engaging the second ratchet gear. A handle is connected to the drive gear for toggle ratchet rotation of the ratchet gears and axle without rotation of the holding gear, to cinch a strap in the slot. Each gear is circular, with an annular series of teeth and with each tooth having a planar surface oriented obliquely to the winding axis, thereby defining low and high edges on each tooth. Springs are provided for imposing an adjustable axial biasing force to the drive gear and holding gear, for selective engagement or disengagement with the ratchet gears. A tourniquet buckle and an industrial buckle are disclosed.

### BACKGROUND

The present invention relates to buckles for securing straps, ranging from medical devices such as tourniquets to industrial devices for securing loads.

U.S. Publication 2010/0049241 discloses a tourniquet of the type wherein a wide strap passes through a buckle having a handle that is cyclically pivotable along the length direction of the strap, with a ratchet mechanism for incrementally tightening the strap around a body part. The buckle has a frame with a cross bar adapted to engage a hook at one end of the strap. The other end of the strap is wrapped around a strap axle supported in the frame and that is rotated by a handle operatively connected to a ratchet mechanism supported in the frame. After placing the strap and buckle around a body part, any slack in the strap is pulled through the buckle and then the handle is cyclically pivoted to incrementally tighten the strap, which remains tightened due to the one-way action of the pawls in the ratchet mechanism.

The tourniquet buckle described in U.S. Publication 2010/0049241 contains a pawl-type ratchet mechanism that is commonly used in a variety of buckles for straps.

### SUMMARY

The buckle according to the present disclosure, relies on a different operating principle and is suitable for use on a variety of straps. Instead of a pawl for implementing the hold feature of the ratchet toggle effect, according to the present disclosure one ratchet gear drives the strap axle and another ratchet gear holds the strap axle. The driving ratchet gear mates with a handle operated drive gear and the other ratchet gear mates with a rotationally fixed holding gear. Each gear in the respective pair of gears mates in a face-to-face relationship. Each of these gears combines two planes per gear tooth: an inclined guide plane and a perpendicular driving plane for one pair and an inclined guide plane and holding plane for the other pair. This produces a smooth ratchet, toggle motion when two mating gears are spring loaded with the inclined planes in a face to face position. The arrangement also enables elective free-wheeling release of the loading with use of an easily accessed nut to disengage and hold apart the mated gears.

This is achieved in that the ratchet mechanism includes a ratchet gear with an annular sequence of wedge-shaped teeth operatively connected to one end of the strap axle and a similarly toothed drive gear in face-to-face confrontation with the ratchet gear. The drive gear is operatively connected to a handle, whereby pivoting the handle in a drive stroke rotates the drive gear, the ratchet gear, and the axle. Similarly shaped confronting gears at the other end of the axle are configured differently, whereby a stationary gear functions analogously to a pawl, by engaging a gear at the other end of the axle to prevent the axle from rotating during the return stroke of the handle. This face-to-face engagement of the gears avoids the complexity and manufacturing cost of installing conventional pawls. An axially loaded spring imposes an axial biasing force to the gears to keep them engaged during cinching of the strap.

In a more specific implementation focused on the gears, the improvement to a strap buckle having a strap axle mounted in a frame with at least one gear operatively connected between a handle and the axle for toggle ratchet rotation of the axle about a winding axis, can be characterized as comprising first and second ratchet gears fixed at respective first and second ends of the axle for corotation with the axle. A drive gear engages the first ratchet gear and a rotationally fixed holding gear engages the second ratchet gear. A handle is operatively connected to the drive gear for toggle ratchet rotation of the first ratchet gear and axle without rotation of the holding gear. Each gear is circular, with an annular series of teeth and with each tooth having a planar surface oriented obliquely to the winding axis, thereby defining low and high edges on each tooth. The high edge of each tooth is a planar surface that extends perpendicularly to the winding axis, from the low edge of an adjacent tooth.

In a first preference, a circular ratchet gear is fixed at each end of the strap axle for corotation with the strap axle, with an annular series of teeth within the circumference. A drive shaft is coaxially aligned with a respective end of the strap axle and supported with freedom to move axially. Each drive shaft has an inner end, with a circular drive gear with an annular series of teeth within the circumference formed for ratcheted engagement with the teeth on the ratchet gear. The ratchet gear and the drive gear each has an annular series of teeth within the circumference, with each tooth having a planar surface oriented obliquely to the winding axis.

In a second preference, an adjustment mechanism is operatively associated with the drive member for imposing a variable biasing force to selectively engage and disengage the drive member with the ratchet gear, so that the strap can be easily loosened and re-tightened.

Another significant feature is the ability of strap material to return to its original size after being compressed momentarily. This interference fit memory utilization has been applied in the product design to eliminate the need for snap tongues, keepers, and similar assembly retention features. The unique positioning of the hook slot opposite the narrowest point of hook provides the location for an interference fit.

### BRIEF DESCRIPTION OF THE DRAWING

A representative embodiment of the invention is described below with reference to the drawing, in which:

FIG. 1 is a perspective view of the buckle portion of the tourniquet according to an embodiment of the invention;

FIG. 2 is an exploded view of the buckle of FIG. 1;

FIG. 3 shows how one end the strap passes over a pad connected to a first cross bar of the buckle, through a slot in the axle, and out of the buckle, and how the other end of the strap hooks onto a second cross bar of the buckle;

FIGS. 4A and 4B schematically show how the teeth on the faces of the ratchet gear and mating drive gear are preferably in the form of an annular series of inclined planar surfaces oriented obliquely to the winding axis, when engaged for ratcheting and disengage for release;

FIG. 5 is a perspective view of the hook at the other end of the strap;

FIG. 6 depicts how the other end of the strap is connected to the hook to help retain the cross bar within the hook during assembly of the tourniquet on the body part;

FIG. 7 shows another embodiment that is adapted for industrial strapping, corresponding to the view of FIG. 1;

FIG. 8 is a schematic cross section through the center of the axle, showing the relationship of the straps to the industrial buckle of FIG. 7; and

FIG. 9 shows the buckle of FIG. 7, in an exploded view corresponding to FIG. 2.

### DETAILED DESCRIPTION

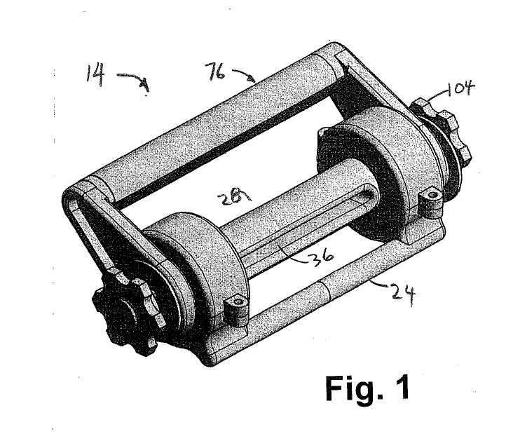

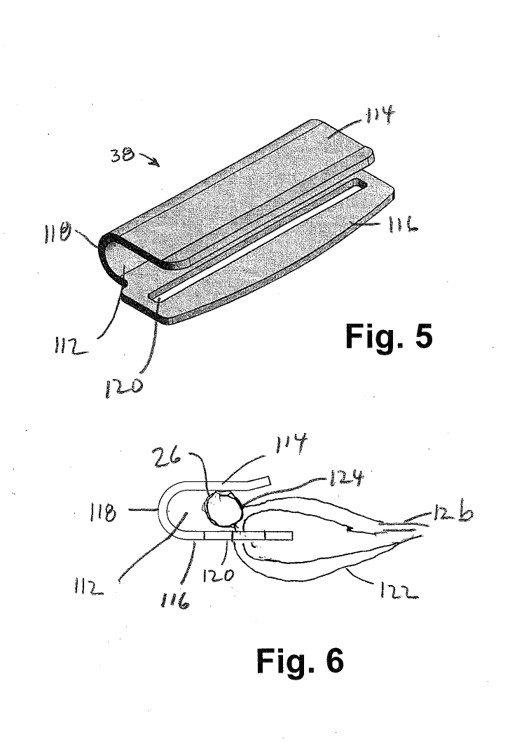

FIGS. 1-3 show the preferred tourniquet 10 according to an embodiment of the invention, for a strap 12 with first and second ends 12a, 12b, having a length of at least about two feet and a width of at least about 1½ inches, preferably about two inches. The strap 12 (shown as a cross-hatched line) passes through a ratchet buckle 14 embodying a rigid frame with two parallel side walls 16, 18 oriented in the length direction of the strap and separated by a distance greater than the width of the strap, each sidewall having a base 20 and a top 22. First and second parallel cross bars or tie rods 24, 26 are supported between the bases of the sidewalls, and spaced apart in the length direction of the strap. A strap axle 28 has first and second ends 30, 32 journaled at the sidewalls for rotation about a winding axis 34, and an axially extending through slot 36, sized to permit free parallel sliding of the strap transversely through the strap axle while when the second cross bar 26 is trapped in a hook 38 (FIG. 5) at the second end 12b of the strap. The slot has four axially extending edges, only two of which 36a, 36b, are shown but with corresponding edges hidden at the rear. A ratchet gear 40, 42 is fixed on the ends of strap axle 28 for co-ration with the axle.

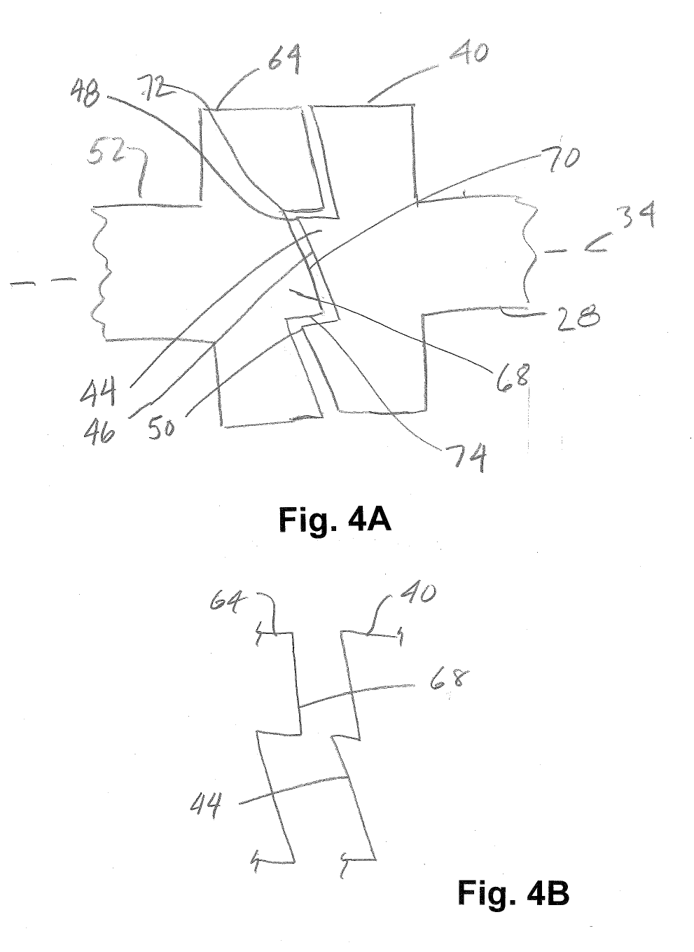

A mating gear is provided for each ratchet gear, forming two operational pairs, only one of which is schematically depicted in FIG. 4. Drive ratchet gear 40 features an annular series of wedge-like teeth 44 within the circumference, each tooth having a planar surface 46 oriented obliquely to the winding axis 34, extending between low 48 and high perpendicular edges 50. Each high edge surface is on a plane that has a height parallel to the axis and a length that is perpendicular to axis 34. When the face of each gear is viewed along the axis, each tooth appears pie-shaped.

A drive shaft 52 and a holding shaft 54 are coaxially aligned with a respective end of the strap axle and are supported on respective side walls 16, 18 with freedom to move axially. Each shaft has an inner end 56, 58 adjacent a respective ratchet gear 40, 42 and an outer end. For the drive stroke, drive gear 64 rotates clockwise and holding gear 66 rotates counterclockwise. Circular drive and holding gears 64, 66 are fixed to the inner end of a respective drive shaft.

Drive gear 64 also has an annular series of teeth 68 within the circumference, with each tooth on drive gear having a planar surface 70 oriented obliquely to the winding axis, thereby defining low and high edges 72, 74 on each tooth. The teeth 68 are the same size and shape as the teeth 44 of the mating ratchet gear 40. Drive gear 64 actively rotates ratchet gear 40 and thus the axle, as a substantially planar high edge 72 of each tooth 64 pushes against a mating substantially high edge planar edge 50 of each tooth on ratchet gear 40.

The holding ratchet gear 42 and mating gear 66 are configured in a manner corresponding to the mated pair 64, 40. During such axle rotation, holding gear 66 is stationary as holding ratchet gear 42 rotates and the inclined tooth surfaces on gear 42 slide on the inclined surfaces of gear 66 until opposed edges mate. Gear 66 functions analogously to a ratchet pawl, whereby its tooth edges hold against the mating tooth edges of ratchet gear 42 while the drive gear 64 is reversed into position for the next incremental advance of the ratchet gear 40.

Thus, during the drive stroke the high edge of each tooth on the drive gear 64 engages and pushes on the high edge of a tooth on the first ratchet gear 40 while the high edge of each tooth on the second ratchet gear 42 slides on the planar surface of a tooth on the holding gear 66 until the high edge of each tooth on the ratchet gear engages the high edge on a tooth of the holding gear. During the retraction stroke the holding gear 66 prevents the second ratchet gear 42, strap axle 28, and first ratchet gear 40 from rotating while the high edge of each tooth on the drive gear 64 slides along the planar surface of a tooth on the first ratchet gear 40 until the high edge of each tooth on the drive gear 64 engages a high edge on a tooth of the first ratchet gear 40 to thereby reset a drive stroke.

The handle 76 extends transversely from and is operatively connected to the drive shafts 52, 54, for rotating the drive shaft 52 while sliding around shaft 54, with freedom for the shafts to move axially. The handle extends at an angle from the frame, and is actuated for the drive stroke by pivoting 78 away from the second cross bar 26.

Means 80, 82 are provided at the outer end of each drive shaft for imposing an axial biasing force to the gears 64, 66. Preferably, the biasing means are adjustable, to selectively engage and disengage the drive and holding gears with the ratchet gears, as shown in FIGS. 4A and 4B. When the shaft gears 64, 66 and ratchet gears 40, 42 are engaged, cyclic pivoting of the handle 76 in direction 78 drives the upper edge 74 of the drive gear teeth against the lower edge 48 of the ratchet gear teeth with resulting incremental rotation of the strap axle 28 commensurate with the distance between the high and low edges of each tooth. During the return stroke of the handle and “hold” of the axle and ratchet gear 42 while the strap has been tightened, the drive gear reverses rotation with planar surface 70 smoothly sliding along planar surface 46. To loosen the strap, the shaft gears 64, 66 and ratchet gears 40, 42 are entirely disengaged so the strap axle 28 can rotate freely. This greatly facilities the tightening and loosening of the tourniquet at the desired 20-minute intervals.

To facilitate the ease of engaging and disengaging the confronting face-to-face gears and thus tightening and loosening of the tourniquet, the strap axle 28 extends axially 84 for rotational journaling within a bore 86 in each shaft 52, 54 or respective gear 64, 66. The handle has spaced-apart drive arms 88 with respective sockets 90a, 90b. Covers 92 have respective recesses 94a and 94b. Socket 90a and cover recess 94b have full and semi-circular profiles, respectively. Socket 90a engages hexagonal profile 96 on drive shaft 52 whereas semi-hexagonal profile 94b engages hexagonal profile 98 holding shaft 54. The former actively rotates the drive and ratchet gears 64, 40 and axle 28, whereas the latter holds the holding gear 66 during rotation of the shaft and ratchet gear 42. Sleeve 102 is provided only to accommodate the idle rotation of socket 90b, which rides over the shank portion of the sleeve.

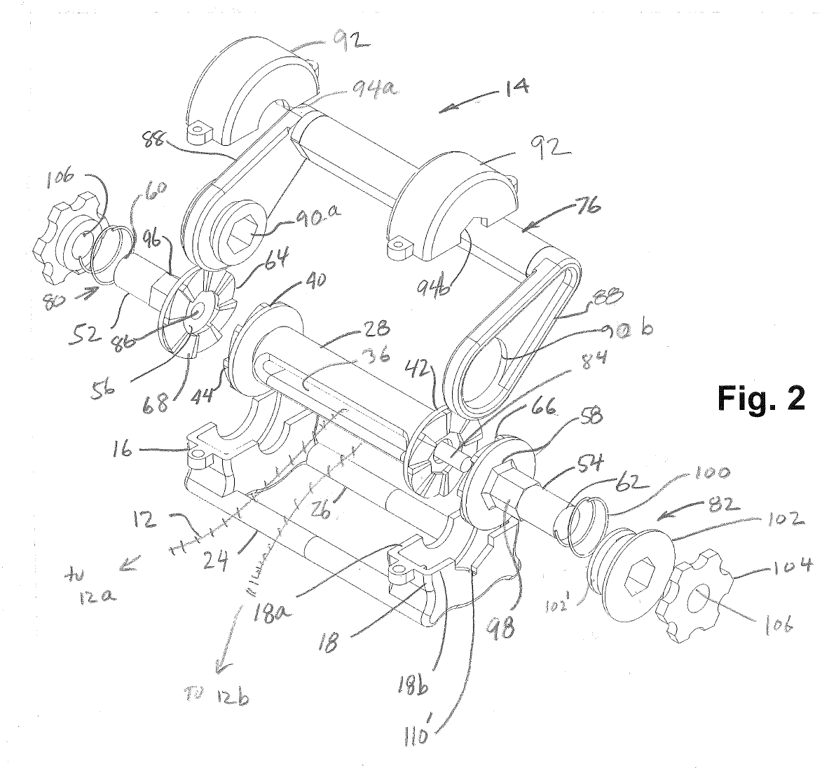

Each biasing means such as 82 shown in FIG. 2 is similar, and as representative, includes a coil spring 100 over the threaded shaft 54 with one end seated against the outside 58 of the drive or holding gear and the other end seated in a slot 102′ on the shank of sleeve 102. The sleeve is internally profiled to mate with the hexagonal profile 98 of the holding shaft 54 while leaving internal space for threaded mating of the thumb wheel or nut 104. The internal threads 106 of the nut engage the threads on shafts 54. The sleeve 102 is not needed on the other side of the axle, where the spring 100 seats on the thumb nut.

Preferably, each side wall 16, 18 has an inner and an outer sub-wall such as 18a, 18b, with a cut out 110 at the top of each sub-wall. The cutouts in both sub-walls of wall 16 and the inner sub wall 18a are semi-circular, but cutout 110′ in outer subwall 18b is semihexagonal to mate with the hexagonal profile 98 on drive shaft 54. The strap axle 28 is supported on the inner sub-walls 18a and the drive shaft is supported by the outer sub-wall 18b. When observing FIG. 2 from right to left, the order of assembled components is thumb nut 104, sleeve 102, handle portion defining opening 90b, subwall 18b, gear 66, and gear 42.

The nuts 104 compress the springs at both sides of the axle. In this embodiment, clockwise rotation of the nuts 104 pulls the respective shafts away from the respective the axle extensions 84, thereby separating the drive gear 64 and holding gear 66 from the respective ratchet gears 40 and 42. It should be appreciated that the configuration for biasing in normal operation and selective relaxation or adjustment of the biasing for disengagement of the gears, can be implemented in other ways.

As shown in FIGS. 5 and 6, the hook 38 attached to one end 12b of the strap defines a generally “U” or “C” shaped channel 112 that has two legs 114, 116 extending from a curved base 118, with the legs and base sized to receive and seat the second cross bar 26 against the base. One leg 116 has a transverse slot 120 spaced from the base such that when the second bar is seated at the base 118 the second bar is situated between the slot and the base. The end 12b of the strap is looped 122 through the slot and secured to itself, with a portion 124 of the loop occupying space between the legs sufficient to provide a resilient resistance to inadvertent disengagement of the second bar from the channel when the second bar is dislodged from the seat during application of the tourniquet. The resistance can easily be overcome during intentional assembly and disassembly of the tourniquet.

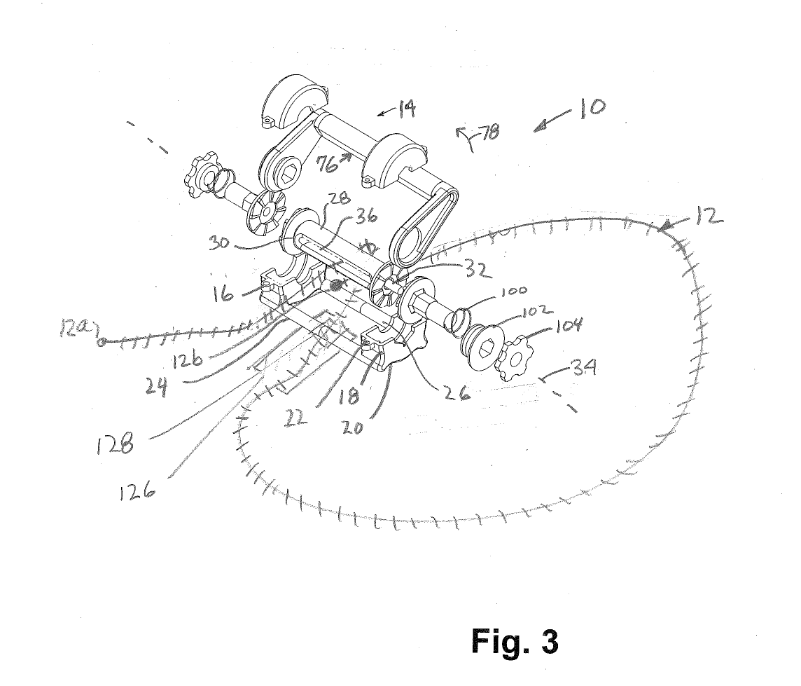

FIG. 3 shows how both ends of the strap are connect to the buckle, with a pad 126 having a down side for bearing on a body part and a top side that is connected with a loop 128 to the first cross bar 24.

FIG. 3 also shows the path for connecting the strap 12 and buckle 14. In the stored condition, the strap region adjacent to the first end 12a of the strap is in the loop 128 of the pad 126 and the strap passes under the strap axle 28 toward the second cross bar 26, then through the slot 36 toward the first cross bar 24 and over the pad. The second end 12b of the strap with hook 38 is free. When the tourniquet is to be applied to a body extremity, for example a thigh, the pad 126 is placed at the front of the thigh and the hook 38 at the second end of the strap is pulled around the thigh and connected to the second cross bar 26. As explained above, the hook is mildly self-retained, while the first end 12a of the strap is pulled through the slot in the strap axle, thereby removing the slack in the strap. The handle 76 can then be actuated, with each pivotal displacement stroke away from the strap producing a commensurate rotation of the drive gears and tightening of the strap. As with any ratchet mechanism, the return stroke (toward the strap) does not affect the strap. The drive and return strokes are cyclically performed until the desired degree of tightening stops the loss of blood.

The strap can be loosened by simply rotating the thumb nuts 104 to separate the drive gears from the ratchet gears, whereby the strap can be pulled in the reverse direction through the slot 36. The tourniquet can be quickly re-tightened by reversing the thumb nuts to re-engage the drive and ratchet gears, and repeating the cyclical stroking of the handle.

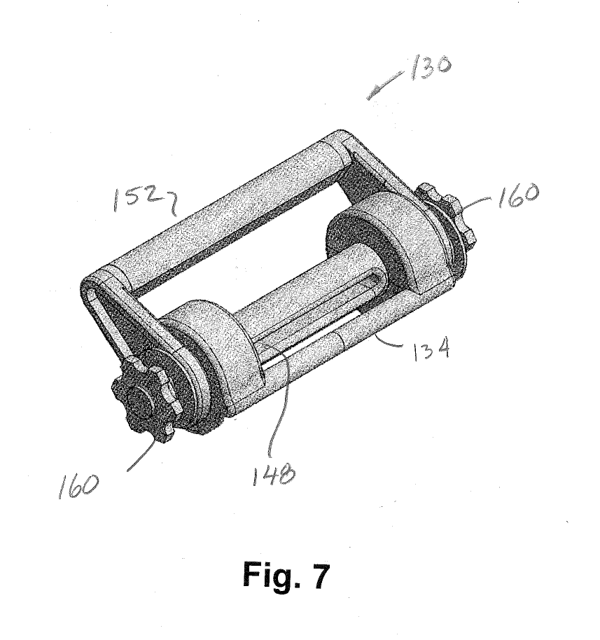

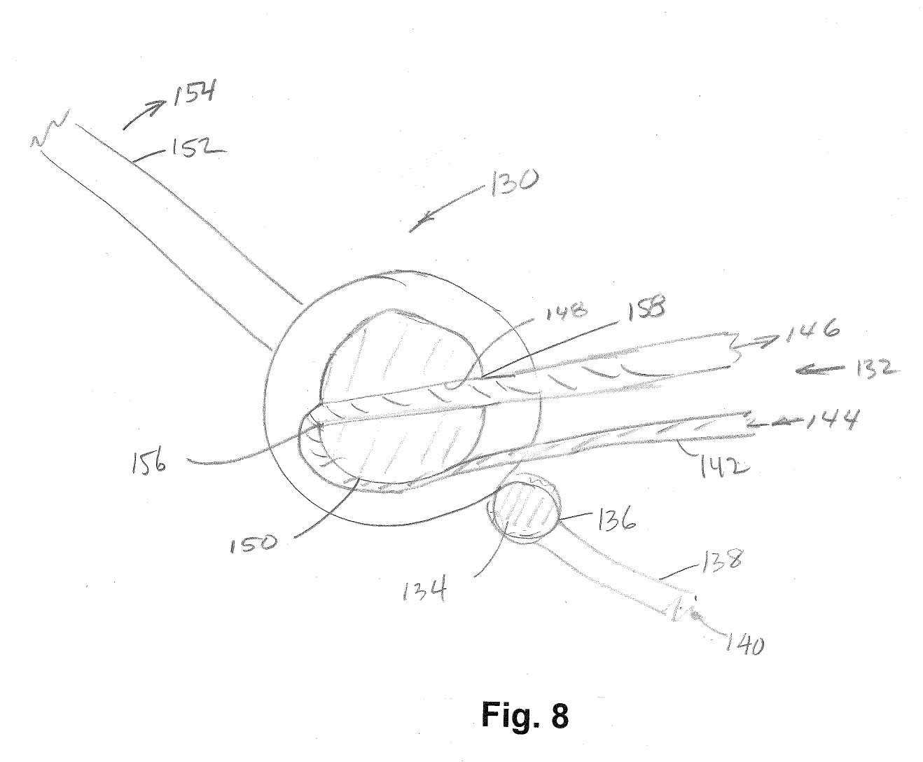

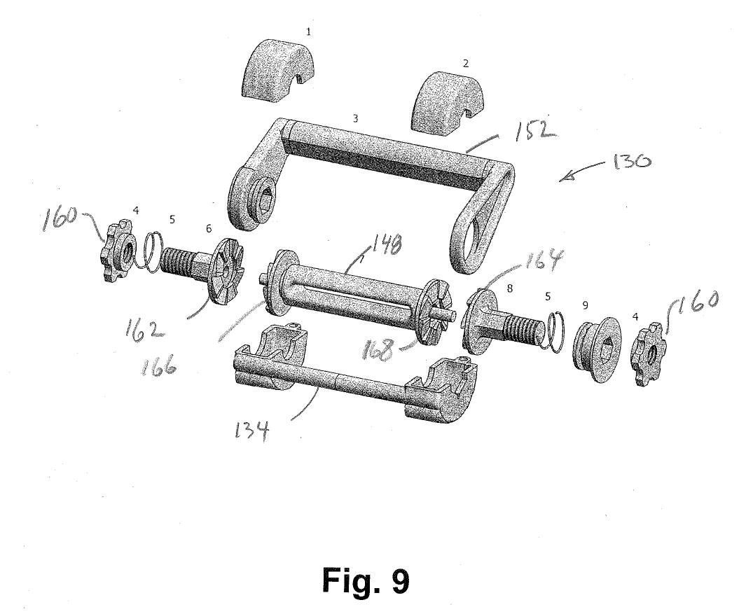

FIGS. 7-9 show another buckle embodiment 130 that is adapted for industrial strapping. FIG. 8 shows the buckle of FIG. 7 with associated strap system 132, and FIG. 9 is an exploded view corresponding to FIG. 2. The main difference relative to the tourniquet buckle of FIGS. 1-6 is that the strap system 132 and buckle 130 need not complete a closed loop when deployed. For this reason only one cross bar or tie rod 134 is required, serving as a mounting bar. One end 136 of a mounting strap 138 or the like is permanently or selectively attached to mounting bar 134, and the other end 140 is, for example, attached to the side of a vehicle flatbed (not shown). Another, securing strap 142 is attached or otherwise secured at one end 144 to the other side of the flat bed, and extends over the load on the flat bed to the buckle 130. The other end 146 of the securing strap 142 passes through the slot 148 of the axle 150 of the buckle 130.

As the handle 152 is driven in the drive stroke 154, the mechanisms and internal gearing operate as described previously and shown in FIG. 8, to rotate the axle 150 whereby the edges 156, 158 advance and tension the strap 142 against the secured end 144 of strap 142, in a ratchet cycle, until the strap 142 tightens down on the load. Also as previously described, the nuts 160 are used to engage or disengage the gears, thereby deploying the ratchet advance mode or release mode, respectively.

1. In a strap buckle having a strap axle mounted in a frame with at least one gear operatively connected between a handle and the axle for toggle ratchet rotation of the axle about a winding axis, the improvement comprising:
first and second ratchet gears fixed at respective first and second ends of the axle for corotation with the axle;
a drive gear engaging the first ratchet gear and a rotationally fixed holding gear engaging the second ratchet gear;
a handle operatively connected to the drive gear for toggle ratchet rotation of the first ratchet gear and axle without rotation of the holding gear;
wherein each gear is circular, with an annular series of teeth and with each tooth having a planar surface oriented obliquely to the winding axis, thereby defining low and high edges on each tooth; and
wherein the high edge of each tooth on each gear is a planar surface that extends perpendicularly to the winding axis, from the low edge of an adjacent tooth of the same gear.

2. The improvement of claim 1, wherein a strap passes through the slot.

3. The improvement of claim 2, wherein the buckle is secured by a stationary mount and said strap is an industrial strap.

4. The improvement of claim 3, wherein the buckle includes a cross bar with connected stationary mount and said strap passes over a load to a remote end that is fixed, whereby ratchet advance of the drive gear tightens the strap over the load.

5. The improvement of claim 2, wherein the strap is a tourniquet strap having one end fixed to the buckle and another end passing through the slot, thereby forming a closed loop, whereby ratchet advance of the drive gear shortens the length of the loop.

6. The improvement of claim 1, wherein the buckle is a tourniquet buckle for tightening a tourniquet strap, wherein:
(a) the frame includes two rigid parallel side walls, separated by a distance greater than the width of the strap;
(b) first and second parallel cross bars are supported between the sidewalls;
(c) the strap axle has opposite ends journaled at the sidewalls for rotation about the winding axis, and an axially extending through slot, sized to permit free sliding of the strap transversely though the strap axle;
(e) a drive shaft and a holding shaft are coaxially aligned with respective ends of the strap axle and supported on respective side walls with freedom to move axially, each shaft having an inner end adjacent a respective ratchet gear and an outer end;
(f) the drive gear is fixed at the inner end of the drive shaft and the holding gear is fixed at the inner end of the holding shaft, each with an annular series of teeth formed for face-to-face ratcheted engagement with the teeth on the first and second ratchet gears, respectively;
(g) the handle extends transversely from and is operatively connected to the drive shaft, for rotating the drive gear, first ratchet gear, strap axle, and second ratchet gear while the holding gear resists rotation;
(h) means at the outer end of each drive shaft for imposing an adjustable axial biasing force to the drive gear and holding gear.

7. A buckle for securing a strap comprising:
a rigid frame including first and second parallel side walls, and at least one cross bar supported between the sidewalls;
a strap axle having first and second ends supported at the respective sidewalls for rotation about a winding axis, and an axially extending through slot;
first and second circular ratchet gears fixed at the respective first and second ends of the strap axle for corotation with the strap axle, each ratchet gear having an annular series of teeth within the circumference;
a drive shaft coaxially aligned with the first end of the strap axle and supported on the first side wall, wherein the drive shaft has an inner end and an outer end;
a circular drive gear fixed to the inner end of the drive shaft, with an annular series of teeth within the circumference, for ratcheted engagement with the teeth on the first ratchet gear;
a holding shaft coaxially aligned with the second end of the strap axle and supported on the second side wall, wherein the holding shaft has an inner end and an outer end;
a circular holding gear fixed to the inner end of the holding shaft, with an annular series of teeth within the circumference for ratcheted engagement with the teeth on the second ratchet gear;
means for imposing an axial biasing force to the drive gear and to the holding gear whereby the teeth on the drive gear mesh with the teeth on the first ratchet gear and the teeth on the holding gear mesh with the teeth on the second ratchet gear;
a handle extending transversely from and operatively connected to the drive shaft for rotating the drive shaft, drive gear, meshed first ratchet gear, strap axle, and second ratchet gear;
means for preventing rotation of the holding gear while the meshed second ratchet gear rotates;
whereby cyclic pivoting of the handle between a drive stroke and a retraction stroke produces incremental ratcheted toggle rotation of the strap axle, with rotation of the axle by the drive gear and first ratchet gear during the drive stroke and holding of the axle against rotation during the retraction stroke as the holding gear holds the second ratchet gear axle, and when the drive gear and ratchet gear are disengaged the strap axle can rotate freely.

8. The buckle of claim 7, wherein
each tooth has a planar surface oriented obliquely to the winding axis, thereby defining low and high edges on each tooth;
during the drive stroke the high edge of each tooth on the drive gear engages and pushes on the high edge of a tooth on the first ratchet gear while the high edge of each tooth on the second ratchet gear slides on the planar surface of a tooth on the holding gear until the high edge of each tooth on the second ratchet gear engages the high edge on a tooth of the holding gear; and
during the retraction stroke the holding gear prevents the second ratchet gear, strap axle, and first ratchet gear from rotating while the high edge of each tooth on the drive gear slides along the planar surface of a tooth on the first ratchet gear until the high edge of each tooth on the drive gear engages a high edge on a tooth of the first ratchet to thereby reset a drive stroke.

9. The buckle of claim 7, including means at the outer end of each shaft for imposing an adjustable axial biasing force to selectively engage and disengage the drive gear and holding gear with the respective ratchet gears; whereby
(a) when the drive gear and holding gear engage the respective ratchet gears, cyclic pivoting of the handle ratchet toggles the axle to drive the drive gear teeth against first ratchet gear teeth with resulting incremental rotation of the strap axle commensurate with the distance between the high and low edges of each tooth, and
(b) when the drive gear, holding gear and ratchet gears are disengaged the strap axle can rotate freely.

10. The buckle of claim 8, including means at the outer end of each shaft for imposing an adjustable axial biasing force to selectively engage and disengage the drive gear and holding gear with the respective ratchet gears; whereby
(a) when the drive gear and holding gear engage the respective ratchet gears, cyclic pivoting of the handle ratchet toggles the axle with resulting incremental rotation of the strap axle commensurate with the distance between the high and low edges of each tooth, and
(b) when the drive gear, holding gear and ratchet gears are disengaged the strap axle can rotate freely.

11. The buckle of claim 8, wherein the high edge of each tooth on each gear is a planar surface that extends parallel and perpendicularly to the winding axis, from the low edge of an adjacent tooth on the same gear.

12. The buckle of claim 10, including a tourniquet strap with one end passing through the slot and another end secured to a cross bar.

13. The buckle of claim 10, wherein
the frame has two cross bars;
a pad is secured to one cross bar;
a tourniquet strap has one end including a hook securable to the other cross and another end passing over the pad and through the slot.

14. A tourniquet comprising:
a strap having a length and a width and extending between opposite ends;
a hook attached to one end of the strap, extending along the width of the strap and defining a channel;
a rigid frame including
(a) two parallel side walls oriented in the length direction of the strap and separated by a distance greater than the width of the strap, each sidewall having a base and a top and
(b) first and second parallel cross bars supported between the base of the sidewalls, and spaced apart in the length direction of the strap;
(c) a strap axle having ends journaled at the sidewalls for rotation about a winding axis, and an axially extending through slot, sized to permit free sliding of the strap transversely though the strap axle while also sliding over the first cross bar when the second cross bar is trapped in the hook at said one end of the strap;
(d) a circular ratchet gear fixed at each end of the strap axle for corotation with the strap axle, with an annular series of teeth within the circumference, each tooth having a planar surface oriented obliquely to the winding axis, thereby defining low and high edges on each tooth;
(e) a drive shaft coaxially aligned with a respective end of the strap axle and supported on a respective side wall with freedom to move axially, each drive shaft having an inner end adjacent a respective ratchet gear and an outer end;
(f) a circular drive gear fixed to the inner end of a respective drive shaft, with an annular series of teeth within the circumference, each tooth having a planar surface oriented obliquely to the winding axis, thereby defining low and high edges on each tooth;
(g) a handle extending transversely from and operatively connected to the drive shafts, for rotating the drive shafts with freedom for the drive shafts to move axially;
(h) means at the outer end of each drive shaft for imposing an adjustable axial biasing force to selectively engage and disengage the drive gears with the ratchet gears; whereby
i. when the drive gear and ratchet gear are engaged, cyclic pivoting of the handle drives the drive gear teeth against ratchet gear teeth with resulting incremental rotation of the strap axle commensurate with the distance between the high and low edges of each tooth, and
ii. when the drive gear and ratchet gear are disengaged the strap axle can rotate freely.

15. The tourniquet of claim 14, wherein the strap axle extends axially for rotational journaling within each drive shaft.

16. The tourniquet of claim 14, wherein the handle has spaced apart drive arms with respective sockets having profiled openings that mate with complementary profiles on the respective drive shafts, with said sockets providing axial stop surfaces operatively associated with said means for biasing.

17. The tourniquet of claim 14, wherein each side wall has an inner and an outer sub-wall, with a cut out at the top of each sub-wall, and the strap axle is supported on the inner sub-wall and the drive shaft is supported by the outer sub-wall.

18. The tourniquet of claim 14, wherein
said channel has two legs extending from a curved base, with the legs and base sized to receive and seat the second bar against the base;
one leg has a transverse slot spaced from the base such that when the second bar is seated the second bar is situated between the slot and the base;
said one end of the strap is looped through the slot and secured to itself, with a portion of the loop occupying space between the legs sufficient to provide a resilient resistance to disengagement of the second bar from the channel when the second bar is dislodged from the seat.

19. The tourniquet of claim 14, wherein a pad has a down side for bearing on a body part and a top side that is connected to the first cross bar.

20. The tourniquet of claim 14, wherein the handle extends at an angle from the frame, and is actuated by pivoting toward the second cross bar trapped by the hook.
